(19) United States
(12) Patent Application Publication
(10) Pub. No.: US 2003/0124581 A1
(43) Pub. Date: Jul. 3, 2003

(54) Newborn screening for hemoglobinopathy by DNA microarray analysis

(76) Inventors: Steven F. Dobrowolski, Park City, UT (US); Zhili Lin, Pittsburgh, PA (US)

Correspondence Address: MCKAY & ASSOCIATES, PC., 801 MCNEILLY ROAD, PITTSBURG, PA, 15226, US

(21) Appl. No.: 10/246,869

(22) Filed: Sep. 19, 2002

Related U.S. Application Data
(60) Provisional application No. 60/324,138, filed on Sep. 21, 2001.

(51) Int. Cl. C12Q 1/68; C12P 19/34

(52) U.S. Cl. 435/6; 435/91.2

(57) ABSTRACT

A method and an associated microarray for detecting hemoglobinopathies by DNA microarray analysis is disclosed for a newborn screening protocol. A fragment of the human beta-globin gene is amplified and immobilized on a glass substrate and is allowed to hybridize with fluorescent dye-labeled oligonucleotide probes matched to either wild type or mutant S, C, and E alleles of the beta-globin gene. The resulting hybridized microarray slide is scanned and analyzed to reveal normal gene sequence or single nucleotide polymorphisms.

### SPECIFIC REFERENCE

This application hereby claims benefit of provisional application serial No. 60/324,138, filed Sep. 21, 2001.

### GOVERNMENT RIGHTS

This invention was made with the United States Government support under Grant No. 8R4HD37757B from the NIH. The United States Government has certain rights in this invention.

### BACKGROUND

Microarrays as defined herein are specially coated glass substrates with specifically defined target DNA immobilized to its surface by chemical interactions. The target DNA is allowed to hybridize with specific fluorescent-labeled nucleic acid probe sequences such that specific alleles of particular genes can be identified. Thus, microarrays can be used to locate DNA sequence polymorphisms, single-nucleotide polymorphisms (SNPs), or small deletions/insertions in a specific chromosome when fluorescent dye-labeled probe DNA is incubated with the tethered target. Terms generally used in steps for microarray production specific for mutation detection are further defined.

There are over fifty mutations in exon 1 of the B-globin gene which are the cause of various hemoglobinopathies. Common mutations include the S (A173T), C (G172A), and E (G232A) alleles. These 3 mutations reside in a 57-nucleotide region in exon 1. While protein analysis remains a standard screening assay, DNA analysis is more sensitive. In fact, transfusion is common in newborns and hemoglobin from transfused red cells interferes with hemoglobin phenotyping. Hemoglobin from transfused cells is often so abundant that the true hemoglobin phenotype is masked. Molecular analysis is unaffected by transfusion because DNA is derived from nucleated cells, but not transfused red cells.

### SUMMARY OF THE INVENTION

It is the objective of the present invention to provide for a method of detecting mutations for hemoglobinopathies using DNA microarray analysis. It is further an objective of the present invention to provide a microarray comprising immobilized targets specific for detecting the mutations responsible for such hemoglobinopathies. The (3) three mutations detected include the S (A173T), C (G172A), and E (G232A, which are missense mutations located on Codon 6 and Codon 26 of the B-globin gene found on chromosome 11.

Accordingly, patient DNA is amplified by PCR. The PCR product is then immobilized onto glass substrates, thereby forming target DNA. All primers used for PCR amplification are designed to be those as set forth in SEQ ID NO: 4 for the E allele forward primer, SEQ ID NO: 5 for the E allele reverse primer, SEQ ID NO: 6 for the S and C allele forward primer, and SEQ ID NO: 7 for the S and C allele reverse primer. Primers are designed such that the melting temperatures (Tm) fall within a 4.5° C. window centering around 61.0° C.

Fluorescent dye-labeled oligonucleotide probes matched to either wild type or mutant S, C, and E alleles of the beta-globin gene were then designed. The probes are those such sequences as set forth in SEQ ID NOs: 8-12, wherein SEQ ID NO: 8 is for E allele mutation detection, SEQ ID NO: 9 is for the E allele wild type, SEQ ID NO: 10 is for the S and C allele wild type, SEQ ID NO: 11 is for the S allele mutation, and SEQ ID NO: 12 is for the C allele mutation. The probes are allowed to hybridize with the target DNA immobilized on the glass substrate, whereby the fluorescent probes will bind only to specific target DNA which is complementary to the probes specific sequence, the binding specificity of which may be dependent on the temperature of the hybridization and any wash steps. Once bound, the fluorescent label enables detection of specific sequences, either mutant or wild type allele, within the target DNA.

Each hybridized microarray slide is then scanned, wherein the fluorescent signal can be detected and an image corresponding to the extent of hybridization may be analyzed. Thus, genetic sequences specific for wild type or mutant alleles for hemoglobinopathies can be analyzed using the present microarray by comparing the fluorescent intensities of each probe after hybridization with target DNA.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a diagrammatic illustration of the process of making a microarray.

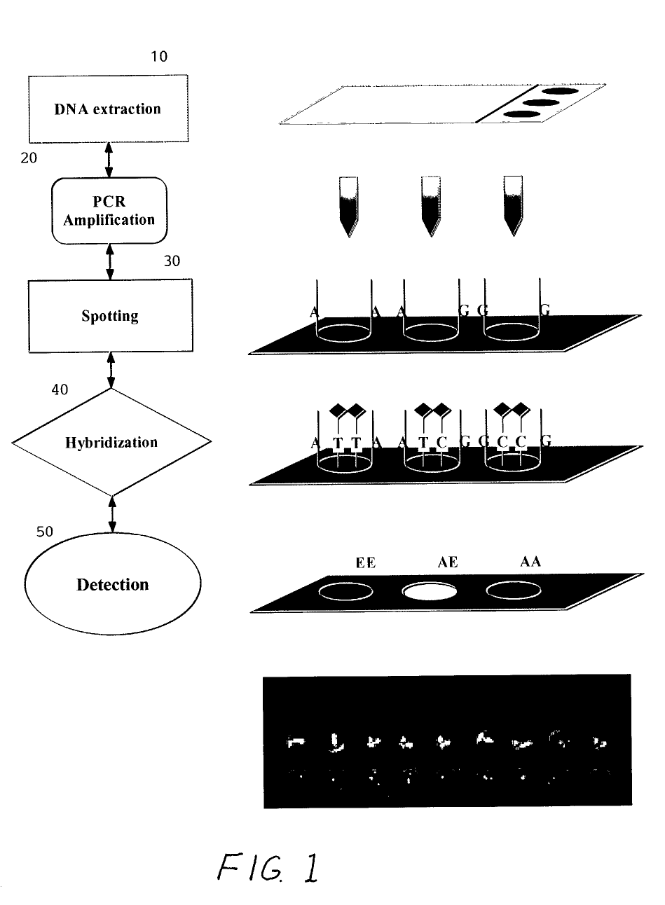

FIG. 2 shows a portion of the human beta-globin gene sequence having the pertinent nucleotide locations for the present invention.

FIG. 3 demonstrates the hybridized microarray slide of the present invention after scanning, revealing the color-image composite.

### DETAILED DESCRIPTION OF THE PREFERRED EMBODIMENT

Definitions:

A “primer” is a short piece of DNA complementary to a given DNA sequence and which acts as the initiation point from which replication proceeds via DNA polymerase (PCR).

“PCR” (polymerase chain reaction) as used herein and as generally known in the art is the rapid technique for amplification of a DNA or RNA sequence, wherein the oligonucleotide primers are annealed (associated) to single stranded nucleotide sequences, which are copied by polymerase.

“Hybridization” defines the process for annealing the complementary sequence through base pairing interaction to the probe.

“Target” is the tethered nucleotide being immobilized to the glass substrate of the microarray to accept the probe (complementary strand) during hybridization.

“Amplicons” may refer to the PCR products, or amplified DNA sequence.

“Microarray” as defined herein is the slide of single strand, immobilized DNA that is adapted to hybridize with labeled probes and undergo image analysis to reveal differences in hybridization patterns.

Hemoglobinopathies, in this embodiment, may be screened using DNA microarray analysis as at least a first tier screening method, and when not used as a first tier screening method, the microarray method may also be coupled to an isoelectric focusing assay and/or genotype analysis using a Lightcycler® instrument. Isoelectric focusing identifies the protein charge differences caused by the S (A173T), C (G172A), and E (G232A) missense mutations as compared to the normal protein charge. Focusing results may then be confirmed by genotype analysis using real-time PCR detection by Florescent Resonance Energy Transfer (FRET) with a Roche Lightcycler® instrument. As is generally known in the art, during FRET, a donor flourophore is excited by an external light source and emits light that is absorbed by a second, acceptor fluorophore. This energy transfer only occurs when two probes are hybridized and in close proximity.

Accordingly, either singularly or as an alternative tier for newborn screening for hemoglobinopathies caused by the genetic missense mutations at S (A173T), C (G172A), E (G232A), a screening method is provided for screening at the molecular level for the above sickle cell hemoglobinopathies.

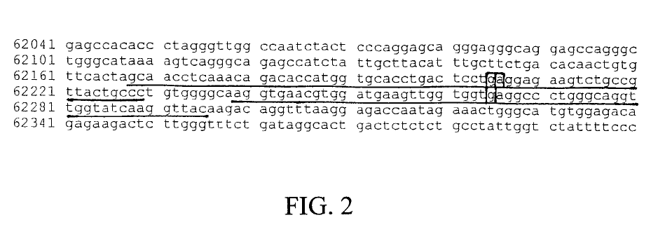

The 3 mutations above reside in the exon 1 region of the human beta-globin gene. The human beta-globin gene sequence (see Genbank accession no. U01317) is used to design primers for the amplification of the S and C alleles in Codon 6 and E allele in Codon 26 and specific probes for detection of these alleles. A portion of the human beta-globin gene sequence having the pertinent nucleotide locations is shown in FIG. 2 (SEQ ID NO: 1). The sequence of the fragment complementary to the sequence analyzed for the S and C allele mutations having nucleotide length of 61 is marked with an underline in FIG. 2, wherein boxes indicate the nucleotide position having the potential mutation (wild type shown). The 61 nucleotide fragment thus analyzed for S (A173T) and C (G172A) mutation detection is then shown by SEQ ID NO: 2. Also marked in FIG. 2 is the sequence of the fragment complementary to the sequence analyzed for the E allele mutation having a nucleotide length of 57 base pairs with an underline at the mutation position (wild type shown). SEQ ID NO: 3 shows the sequence analyzed to determine any mutation at the respective position.

Synthesis of patient specimen amplicons is accomplished by PCR. Genomic patient DNA samples are collected from any traditional methods, such as from any tissue or organ from which RNA or DNA can be amplified, or by purification from a dried blood spot on filter paper. Advanced primer design software may be used to design the primers. In this embodiment, primers are designed such that Tm's fall with a 4.5° C. window centering around 61.0° C. This enables the use of common amplification conditions for all primer pairs. Table 1 below shows the primers developed for the current assay.

TABLE 1

| Seq. | | | |
|---|---|---|---|
| ID | Primer | | Tm |
| No. | Name | Sequence | (° C.) |
| 4 | BGEfwd | 5′ AGGTGAACGTGGATGAAGTT 3′ | 61.1 |
| 5 | BGEMrev | 5′ C6-GTAACCTTGATACCAACCTGC 3′ | 60.6 |
| 6 | BGSCfwd | 5′ GCAACCTCAAACAGACACCA 3′ | 62.6 |
| 7 | BGSCMrev | 5′ C6-GGCAGTAACGGCAGACTTCT 3′ | 64.1 |

A modifier, such as a C6 amino modifier, is attached to the 5′ end of selected primers to enable the attachment and isolation of a specific strand to a treated glass substrate used for microarray printing. Substrates for array printing may vary in terms of cost, background, and DNA binding capacity, and are in this embodiment, essentially a non-porous, glass slide with a chemical coating having a DNA binding surface. For each PCR primer set, only one primer has the C6 amino modifier. By doing this, only one strand of the amplicon will attach to the glass slide, and the other strand will be washed away during processing. Selection of a particular strand acts to enhance the hybridization efficiency by eliminating competition between the complementary amplicon strand and the assay probe for target binding. The PCR products are printed onto the treated slides. As an alternative embodiment they may be purified prior to any printing step. These targets are then ready for hybridization with a complementary probe.

Fluorescent dye-labeled oligonucleotide probes matched to either wild type or mutant S, C, and E alleles of the beta-globin gene were then designed as shown in table 2.

TABLE 2

| SEQ ID | | | Tm |
|---|---|---|---|
| NO. | Probe Name | Sequence | (° C.) |
| 8 | EDETsen1MUT | 5′ Cy5-TTGGTGGTAAGGCCC 3′ | 55.8 |
| 9 | EDETsen2WT | 5′ Cy3-AGTTGGTGGTGAGGC 3′ | 56.2 |
| 10 | SCWTsen | 5′ Cy5-GACTCCTGAGGAGAA 3′ | 50.4 |
| 11 | SMUTsen | 5′ Cy3-GACTCCTGTGGAGAA 3′ | 51.3 |
| 12 | CMUTsen | 5′ Cy3-GACTCCTAAGGAGAAG 3′ | 49.6 |

Hybridization is the process of incubating the immobilized target DNA tethered on the glass substrate with the labeled probe DNA at a particular temperature. The fluorescent probe DNA will hybridize with the primer-amplified target DNA, will be washed at a probe specific temperature, and the amount of immobilized fluorescence can be determined by scanning.

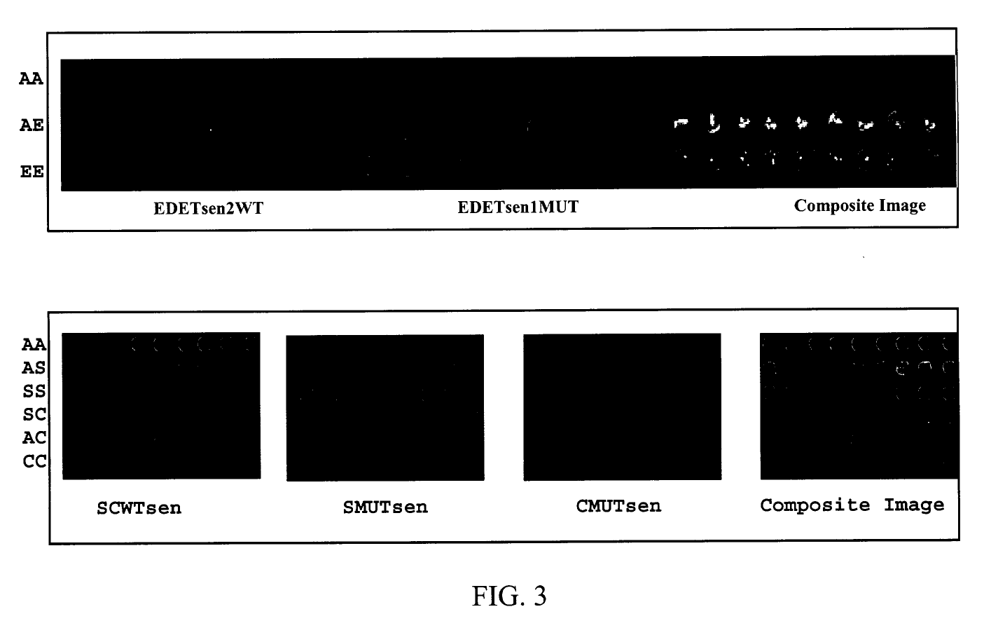

Each spot of DNA contains pixels, which are illuminated one pixel at a time using lasers until all the spots on the DNA chip have been scanned and recorded as a high-resolution image file. The scanned images are analyzed in an automated data extraction process that measures the absolute and relative fluorescence at two wavelengths. The use of the present targets and probes for DNA microarray analysis produces the hybridized microarray slide as seen in FIG. 3, specific for the detection of the wild type and/or genetic missense mutations for sickle cell diagnosis.

### EXAMPLE

Samples and DNA Preparation 10

Hemoglobinopathies are first tier screened using a isoelectric focusing assay. Isoelectic focusing identifies the protein charge differences caused by the S (A173T), C (G172A), and E (G232A) missense mutations as compared to the normal protein charge.

Focusing results are then confirmed by a second tier genotype analysis using real-time PCR detection by Florecencent Resonance Energy Transfer with the Roche LightCycler Instrument.

Genomic DNA of genotypes, which are unknown at the time of patient screening but are known for controls for Wild Type, Heterozygous, and Homozygous, is extracted from Dried Blood Spot (DBS). DNA is extracted from a 3.2 mm punch of DBS and is resuspended in a final volume of 100 ul.

PCR Amplification 20

Synthesis of patient specimen amplicons is accomplished by PCR. Primer Premier 5.0 and Oligo 6.0 are among the most advanced PCR primer design software packages and are both employed for primer design. Primers are designed such that Tm's fall within a 4.5° C. window centering around 61.0° C. This enables the use of common amplification conditions for all primer pairs. All primers are synthesized and HPLC purified by Operon Technologies, Inc. (Alameda, Calif.) (See primer sequence in table 1). A C6 amino modifier is attached to the 5′ end of selected primers to enable the attachment and isolation of a specific strand to the glass substrate used for microarray printing. For each PCR primer set, only one primer has the C6 amino modifier. By doing this, only the one strand of the amplicon will be attached to the glass substrate, the other strand will be washed away during the processing step. Selection of a particular strand acts to enhance the hybridization efficiency by eliminating competition between the complementary amplicon strand and the assay probe for target binding.

The PCR amplification reaction (10 μl) contained 10 mM Tris-HCl; 1.5 mM MgCl₂; 50 mM KCl; 4 μl DNA; 0.5 μM each of primers; 200 μM each of dATP, dCTP, dGTP, and dTTP; 0.08 μg TaqStart Antibody (CloneTech); 0.4 unit Taq Polymerase (Roche). PCR was performed in a MWG Biotech PrimusHT Multibloack thermal cycler. Cycling condition are one cycle at 94 ° C. for 2 min, 40 cycles of 94° C. for 20 sec, 58° C. for 30 sec, 72° C. for 20 sec, and a final cycle at 72° C. for 2 min.

MicroArray Printing and Processing 30

There is no PCR purification needed after the PCR reactions are completed. Ten μl of sodium phosphate spotting buffer (300 mM sodium phosphate, pH 8.5/0.02% SDS) was added to each 10 μl PCR reaction, and then printed onto Eppendorf's CreativeChip Oligo slides (Hamburg, Germany) using Virtek Vision's ChipWriter Professional Arrayer (Waterloo, ON, Canada). Printed slides were incubated at 50° C. for 1 hour in a humidified chamber, baked at 80° C. for 1 hour, incubated with 200 ml boiled dH₂O for 3 min, then dried by centrifigation.

Hybridization 40

Fluorescent dye-labeled oligonucleotide probes matched to either wild type or mutant S, C, and E alleles of the β-Globin gene were designed as indicated in table II, and were synthesized and HPLC purified by Operon Technologies, Inc. (Alameda, Calif.). All probes were analyzed by spectrophotometry for oligonucleotide and fluorophore concentration. The Hybridization solution contains Quantifoil's QMT Hybridization buffer (Jena, Germany) with both wild type and mutant probes at a final concentration of 0.1 μM. Each hybridization reaction was carried out with 20 μl of hybridization solution added to the printed slides, covered with a glass coverslip, sealed in the hybridization cassette with 50 μl of dH₂O added to the humidity control chamber. The cassette was then put into a circulated water bath, incubated at 50 ° C. for 1 hour. After 1 hour incubation, the slide was taken out of the cassette and the coverslip was removed. Excess probe is immediately rinsed from the slide by dipping the slide into a room temperature solution of 2×SSC, 0.1% sarcosyl. Non-specific probe binding is then washed from the slide by placing the slide into a container of 2×SSC, 0.1% sarcosyl and incubated in this solution at 50° C. for 10′. The slides are then further washed by dipping 2 to 3 times in a room temperature solution of 2×SSC followed by dipping 2 to 3 times in a room temperature solution of 0.2×SSC. The slide is then immediately dried by centrifugation for 3′ at room temperature.

Microarray Scanning

Each hybridized microarray slide was scanned with Virtek Vision's ChipReader scanner. For both Cy3 and Cy5 channel of the scanner, laser power was set at 100, detector gain at 1, the number of scans at 1, and detector sensitivity at 1000.

Image Composite

The two images from scanning of each microarray slide was composited with ArrayPro Analyzer (Media Cybernetics, L.P.).

Methods of Data Analysis and Interpretation 50

The resulting data may be analyzed using a variety of approaches of different approaches. For the purposes of the present invention, two examples are herein described, though others may be readily apparent to those of ordinary skill in the art. The first approach is by visual inspection of the resulting wild type and mutant signal composite image as described below. Since the wild type and the mutant probes are labeled with different florophors, each will emit light at a different wavelength. When the slide is scanned at each of these two wavelengths, two images are created. One image corresponds to the wild type probe signal and the other image corresponds to the mutant probe signal. After the images are acquired, analysis software is used to assign each image a specific color. One color will be assigned to the signal resulting from the wild type probe and another color will be assigned to the signal resulting from the mutant probe. When the two images (wild type and mutant) are combined into a single composite image, all possible genotypes, including samples containing any combination of the mutant and wild type alleles (heterozygotes), can be visually detected. This visual detection is possible because samples containing a combination of the E,S, or C wild type or mutant alleles will composite to produce additional computer generated colors which will differ from the colors originally assigned to the wild type and mutant alleles. One will only need to visually determine the sample image color to determine the sample genotype.

A second analysis approach is used to assign quantitative values to each genotype. In this approach, the slides are probed with both wild type and mutant probes of differing florophor emission wavelengths as in the above approach. Also as above, the slide is scanned and a separate image is created for the mutant signal and the wild type signal. From this point, the signal from each of the images is quantified using array analysis software. For each sample, a ratio of wild type to mutant signal is calculated. The resulting quantitative ratios can then be categorized into distinct value ranges for each of the possible genotypes. All of this quantification is performed by computer analysis, genotypes are computer assigned, and results are outputted for easy and rapid interpretation in a high-through-put screening laboratory.

I claim:

1. A method of detecting mutations for hemoglobinopathies residing at the S, C, and E alleles of the human beta-globin gene using DNA microarray analysis, comprising:
amplifying patient DNA to form a PCR product;
immobilizing said PCR product onto a substrate, thereby forming target DNA;
allowing probes to hybridize with said target DNA, wherein said probes are selected from the group consisting of those such sequences as set forth in SEQ ID NOS: 8-12, thereby forming a hybridized microarray slide; and,
scanning said hybridized microarray slide to detect an extent of hybridization of said probes with said target DNA.

2. The method of claim 1, further comprising the step of analyzing data resulting from said extent of said hybridization.

3. The method of claim 2, wherein said data is color image data.

4. The method of claim 2, wherein said data is a quantitative ratio of a wild type to mutant signal.

5. The method of claim 1, wherein said patient DNA is extracted from a dried blood spot on filter paper.

6. The method of claim 1, wherein for the step of amplifying patient DNA, a primer having the sequence as set forth in SEQ ID NO: 4 is used as a forward primer for said E allele.

7. The method of claim 1, wherein for the step of amplifying patient DNA, a primer having the sequence as set forth in SEQ ID NO: 5 is used as a reverse primer for said E allele.

8. The method of claim 7, wherein a C6 amino modifier is attached to a 5′ end of said reverse primer.

9. The method of claim 1, wherein for the step of amplifying patient DNA, a primer having the sequence as set forth in SEQ ID NO: 6 is used as a forward primer for said S and C alleles.

10. The method of claim 1, wherein for the step of amplifying patient DNA, a primer having the sequence as set forth in SEQ ID NO: 7 is used as a reverse primer for said S and C alleles.

11. The method of claim 10, wherein a C6 amino modifier is attached to a 5′ end of said reverse primer

12. The method of claim 1, wherein said probes are labeled with a fluorescent dye.

13. A microarray specific for detecting mutations responsible for hemoglobinopathies, comprising:
a glass substrate;
amplified patient DNA immobilized on said glass substrate; and,
one or more probes hybridized with said patient DNA, wherein said probes are matched to either mutant or wild type alleles of human beta-globin.

14. The microarray of claim 13, wherein said one or more probes has the following sequence: TTGGTGGTAAGGCCC.

15. The microarray of claim 13, wherein said one or more probes has the following sequence: AGTTGGTGGTGAGGC.

16. The microarray of claim 13, wherein said one or more probes has the following sequence: GACTCCTGAGGAGAA.

17. The microarray of claim 13, wherein said one or more probes has the following sequence: GACTCCTGTGGAGAA.

18. The microarray of claim 13, wherein said one or more probes has the following sequence: GACTCCTAAGGAGAAG.

19. A fluorescent dye-labeled oligonucleotide probe matched to either wild type or mutant S, C, and E alleles of the human beta-globin gene, comprising a sequence selected from the group consisting of those such sequences as set forth in SEQ ID Nos: 8-12.


SEQUENCE LISTING (12 sequences)

SEQ ID NO: 1 - DNA, 360 bp, Homo sapiens
gagccacaccctagggttggccaatctactcccaggagcagggagggcaggagccagggctgggcataaaagtcagggcagagccatctattgcttacatttgcttctgacacaactgtgttcactagcaacctcaaacagacaccatggtgcacctgactcctgaggagaagtctgccgttactgccctgtggggcaaggtgaacgtggatgaagttggtggtgaggccctgggcaggttggtatcaaggttacaagacaggtttaaggagaccaatagaaactgggcatgtggagacagagaagactcttgggtttctgataggcactgactctctctgcctattggtctattttccc

SEQ ID NO: 2 - DNA, 61 bp, Homo sapiens
ggcagtaacggcagacttctcctcaggagtcaggtgcaccatggtgtctgtttgaggttgc

SEQ ID NO: 3 - DNA, 57 bp, Homo sapiens
gtaaccttgataccaacctgcccagggcctcaccaccaacttcatccacgttcacct

SEQ ID NO: 4 - DNA, 20 bp, Homo sapiens
aggtgaacgtggatgaagtt

SEQ ID NO: 5 - DNA, 20 bp, Homo sapiens
gcaacctcaaacagacacca

SEQ ID NO: 6 - DNA, 21 bp, Homo sapiens
gtaaccttgataccaacctgc

SEQ ID NO: 7 - DNA, 20 bp, Homo sapiens
ggcagtaacggcagacttct

SEQ ID NO: 8 - DNA, 15 bp, Homo sapiens
ttggtggtaaggccc

SEQ ID NO: 9 - DNA, 15 bp, Homo sapiens
agttggtggtgaggc

SEQ ID NO: 10 - DNA, 15 bp, Homo sapiens
gactcctgaggagaa

SEQ ID NO: 11 - DNA, 15 bp, Homo sapiens
gactcctgtggagaa

SEQ ID NO: 12 - DNA, 16 bp, Homo sapiens
gactcctaaggagaag